(19) United States
(12) Patent Application Publication
(10) Pub. No.: US 2021/0393397 A1
(43) Pub. Date: Dec. 23, 2021

(54) INTRAOCULAR IMPLANT

(71) Applicant: Implandata Ophthalmic Products GmbH, Hannover (DE)

(72) Inventors: Max Ostermeier, Seevetal (DE); Stefan Meyer, Hannover (DE); Burkhard Dick, Bochum (DE)

(73) Assignee: Implandata Ophthalmic Products GmbH, Hannover (DE)

(21) Appl. No.: 16/345,542

(22) PCT Filed: Oct. 27, 2017
(86) PCT No.: PCT/US2017/077697

§ 371 Date: Apr. 26, 2019

(30) Foreign Application Priority Data
Oct. 28, 2016 (DE) 102016221368.7

(51) Int. Cl. A61F 2/16; A61B 3/16

(52) U.S. Cl. CPC A61F 2/1608; A61B 3/16; A61F 2002/1681; A61B 2562/0247; A61F 2210/0071; A61F 2250/0002; A61F 2250/006; A61F 2210/0023

(57) ABSTRACT

The invention relates to an implant for implanting in and/or an eye using fixing means enabling improved fixing, in particular with respect to the natural variability of the anatomical conditions of the eye. According to the invention, the fixing means comprise at least one support element which can be mounted on a tissue structure in the direction traverse to the optical axis of the eye.

### CROSS-REFERENCE TO RELATED APPLICATIONS

This patent application is the US National Phase Under 371 of International Patent Application No. PCT/EP2017/077697, entitled “INTRAOCULAR IMPLANT,” naming as inventors Max Ostermeier, Stefan Meyer, and Burkhard Dick, and filed Oct. 27, 2017, which application claims priority to German Patent Application No. 102016221368.7, filed Oct. 28, 2016, which patent documents are incorporated by reference herein in their entireties and for all purposes.

The present invention relates to an implant for implanting in and/or on an eye using fixing means.

Implants of the type mentioned at the outset are known from the prior art, for example, as disc rings, which are fixed by means of fixing means, namely plate-shaped tabs, mostly in the ciliaris sulcus of the eye, after implanting.

In this case, the implants are often equipped with a sensor for measuring intraocular pressure and a telemetry unit for transmitting the measured signal detected by the sensor, such as an induction coil arranged in the plane of the disc ring.

Implants of this type are fixed in the implanted state with respect to the optical axis of the eye, i.e. the axis defined by the axis of rotational symmetry of the pupil, in that the tabs protrude into the ciliaris sulcus. However, disadvantageously, the eye has to be subjected to complex measurements before implanting one of the known implants, so that an implant suitable for fixing can be selected based on the size of the disc ring diameter associated with the implant. This is due to the fact that the known implants virtually provide no adaptation to the naturally occurring variations in anatomical conditions, for example the diameter spectrum defined by the ciliaris sulcus in patients. Specifically, there is therefore a risk in known implants that they are poorly fixed, if their diameter was not determined prior to implanting with respect to the fixing on the ciliaris sulcus, for example.

In addition, due to the annular shape of known implants and the typically rigid tabs relatively narrow limits are disadvantageously imposed with respect to an optimal dimensioning for the fixing. On the one hand, the tabs must not be extended arbitrarily in the plane of the disc ring, as this causes dimensional instabilities in the implant, in particular a potentially tissue-damaging dent. On the other hand, it is required that the tabs have a minimum length in the plane of the disc ring to ensure fixing, since the implant would otherwise be insufficiently fixed.

The danger of a defective fixing associated with the known implants can have fatal consequences for a patient. In the case of defective fixing, migration and/or rotation of the known implants may occur after implanting, in that they and, if present, the sensor attached thereto, together with the telemetry unit, penetrate the eye tissue and/or the iris. Possible consequences are an increase in intraocular pressure, defects in the iris or a shadowing the optical axis of the eye.

Against this background, the object of the present invention is thus to provide an implant of the type mentioned at the outset which, by overcoming the disadvantages of the prior art, allows an improved fixing, in particular with regard to the naturally occurring variability of the anatomical conditions of the eye.

This object is achieved in a generic implant in that the fixing means comprise at least one support element, which can be mounted on the tissue structure in the direction transverse to the optical axis of the eye. Advantageously it is thus ensured that the implant may be fixed transversely to the optical axis of the eye, for example, non-positively by means of, for example, a biasing force and/or by means of at least one positive connection between the support element and the tissue structure. In this case, a ‘tissue structure’ refers to any structure that is capable of fixing the implant according to the invention with respect to the optical axis of the eye via the fixing means, which may for example consist of pressure-elastic springs having arcuate portions. This is in particular the structure which is formed in the posterior chamber of the eye by the anatomical furrow between the ciliaris body and the iris root, i.e. the ciliaris sulcus, so that the support element, which may for example be in the form of an arcuate portion of a compression spring, rests at the bottom of this anatomical furrow, for example by contacting the furrow bottom. In this way, advantageously, a migration and/or rotation of the implant according to the invention is avoided because the implant according to the invention may be fixed with respect to the optical axis of the eye, and in particular transversely to the optical axis of the eye. Advantageously, the implant according to the invention may be fixed via the support element, such as a portion of a surgical thread, but also on a tissue structure in the anterior chamber of the eye, namely preferably in the iridocorneal chamber angle. This also advantageously ensures, in such an implanting, a fixing of the implant according to the invention sufficient for minimising tissue and/or iris damage.

In a preferred embodiment of the implant according to the invention, the fixing means comprise at least one deflecting element for deflecting the support element in a deflection plane, the deflection plane being positioned in the implanted state perpendicular to the optical axis of the eye. Advantageously, by means of the deflecting element, which may be configured, for example, as an adjusting element in the form of an adjustment screw or as an elastic spring, a deflection of the support element transversely to the optical axis of the eye may be achieved, so that the fixing of the implant according to the invention to be provided by the support element, such as the support of a bow portion of a compression spring on the furrow bottom of the ciliaris sulcus, for example, may cover the naturally occurring diameter spectrum of the ciliaris sulcus. This means that, by means of the deflecting element, the implant according to the invention is suitable, for example, for fixing in patients with a relatively small ciliaris sulcus as well as in patients with a relatively large ciliaris sulcus. In practice, therefore, the storage of differently sized implants is advantageously avoided, thus reducing the logistical effort and associated costs both on the part of the eye hospital and the manufacturer. Since the deflection plane in the implant according to the invention is also positioned perpendicular to the optical axis of the eye, the occurrence of torques negatively affecting the positioning of the support element and thus the fixing is advantageously minimised in the implant according to the invention, whereby the implant according to the invention is advantageously subject mainly to reaction forces in the deflection plane.

Another preferred embodiment of the implant according to the invention provides that the deflecting element is able to recover its shape after deformation of the deflecting element, wherein the deflection is based on the shape recoverability. Due to the shape recoverability, i.e. the property of the deflecting element to undergo dynamic shape modification tending towards a non-deformed state, such as in the case of the unloading of a prestressed elastic spring, the deflecting element, i.e., for example, an elastic compression spring having arcuate portions, may unfold, advantageously automatically, i.e. without or almost with no intervention by a physician performing the implant, so that the support element, such as an undulating portion of a spring, transversely moves in the direction of the optical axis, for example, to the furrow bottom of the ciliaris sulcus until a contact is formed between the support element and the furrow bottom of the ciliaris sulcus. Due to the shape recoverability of the deflecting element, it is advantageously also possible that the implant according to the invention may be implanted in a particularly simple and secure way using a small-scale cutting technique by means of a relatively simple or standard injector for intraocular lenses (parameters of the clear corneal technique, <3.5 millimetres or preferably <3 millimetres or more preferably <2.8 millimetres or <2.5 millimetres). The advantages associated with the implant according to the invention also manifest themselves in the fact that the shape recoverability of the deflecting element, i.e., for example, the deflection of a deformed spring consisting of a polymer having shape memory properties, allows a nearly universal adaptation of the support element, such as for example a bow-shaped portion of a spring, to the anatomical conditions of the eye, in particular the diameter of the ciliaris sulcus, after a certain switching temperature has been exceeded. A preoperative dimensioning with regard to the fixing of the implant according to the invention is therefore advantageously eliminated. This is important, since even hospitals with modern facilities often do not have the measurement apparatus necessary for an exact measurement of the anatomical conditions of the eye, such as the diameter of the ciliaris sulcus. In addition, methods for estimating the anatomical conditions on the basis of biometric data are often subject to errors and thus are generally unsuitable. Accordingly, the implanting of the implant according to the invention is advantageously associated only with a low operative, especially preoperative, effort.

Preferably in the implant according to the invention, it is provided that the shape recoverability is elastic. Thus, the deflecting element may be, for example, a mechanical spring. Advantageously for the fixing of the implant according to the invention, this means that an elastic biasing force caused by the spring in the furrow bottom of the ciliaris sulcus, for example, ensures a local support of the support element, i.e., for example, the portion of a compression spring having a relatively large radius of curvature, transverse to the optical axis of the eye.

An implant according to the invention may also be designed such that the shape recoverability may be stimulated via a stimulus. Advantageously for the implanting of the implant according to the invention, the shape recoverability can therefore be switchable. A ‘stimulus’ accordingly refers to any external stimulus that causes a shape memory effect in a suitable material. Consequently, the deflecting element of the implant according to the invention can advantageously be implanted in a deformed state such that, after a physician performing the implanting has found a suitable position for fixing the implant according to the invention, such as the ciliaris sulcus, the deflecting element unfolds in a manner advantageous for fixing, due to a shape memory effect produced by a stimulus, such as a UV light pulse.

In a development of the invention, the stimulus is a variation of temperature. This is a stimulus which is particularly easy to implement and thus particularly advantageous with regard to minimising the operational effort, through which a shape memory effect can be optionally caused, for example, in metals and polymers. For example, a spring made of a shape memory polymer may serve as a deflecting element in the implant according to the invention. If the shape memory polymer is heated above a composition-specific temperature, a shape recovery of the spring takes place, i.e. a deflection of the exemplary deflecting element such that its support element contacts, for example, the furrow bottom of the ciliaris sulcus. Obviously, the shape memory effect known from metals is also advantageously applicable in that the deflecting element of the implant according to the invention consists of such a metal.

Preferably, the deflecting element and/or the support element consist of a polymer. This is advantageous in terms of a fixing, which is as trauma-free as possible, of the implant according to the invention because a large number of polymers have a stiffness suitable for the implanted state, i.e. a modulus of elasticity, which provides the deflecting element, such as a polymer thread, and/or the support element, such as a portion of the polymer thread, with dimensional stability together with an elastic yield, which does not damage the eye tissue.

In the implant according to the invention, if the deflecting element and/or the support element are composed at least in part of a material having shape memory properties, the unfolding of the deflecting element is particularly advantageous. This is due to the fact that the implant according to the invention can advantageously be introduced, for example, into a tube-like injector for implanting the implant according to the invention. After exiting the injector at a position intended for implanting, such as, for example, the ciliaris sulcus, the deflecting element, such as a thread made of a shape memory polymer, unfolds on the basis of a shape memory effect in accordance with a shape intended for the fixing of the implant, such as a curved shape, so that the support element is supported, for example, on the furrow bottom of the ciliaris sulcus.

The term ‘shape memory properties’ or ‘memory effect’ refers to any material property of a metal or polymer, which, due to a phase transition or a modification of the chemical crosslinking of polymer chains, allows a shape change of the deflecting element on a macroscopic scale, in particular in the millimetre or centimetre order, starting from a deformed state of the deflecting element.

According to a variant of the invention, the deflecting element is rigidly formed with respect to dents in the direction perpendicular to the deflection plane. With respect to a fixing, which is as trauma-free as possible, of the implant according to the invention, this ensures that the deflecting element, i.e. a compression spring, the elastic portions of which are in the deflection plane, for example, do not bulge in the implanted state along the optical axis of the eye, which may possibly lead to injuries and long-term trauma of the iris and other surrounding tissues.

In a further advantageous embodiment of the implant according to the invention, it is provided that the deflecting element and/or the support element are designed to be arcuate at least in portions. This has a positive effect on the preferred shape recoverability of the deflecting element for implanting the implant according to the invention, or the elastic flexibility of the support element required for injury-free implanting, for example the arcuate portion of a compression spring made of a shape-memory polymer. In this case, deflecting elements and/or support elements having C- and/or Z-shaped portions lying in the deflection plane are particularly preferred.

In a preferred development of the implant according to the invention, the support element has a segmented surface on a surface provided for contacting with the tissue structure, such as the surface of an arcuate portion of a compression spring. Thus, it is advantageously ensured for the fixing of the implant according to the invention that the support element rests at least in portions, with a form-fit, on the typically segmented surface of the tissue structure such as the ciliaris sulcus. The surface structure of the support element can therefore have, for example, steps, the surfaces of which abut against the step surfaces of the ciliaris sulcus formed by membranous segmentations in a tangential direction to the approximately circular periphery defined by the ciliaris sulcus, whereby the implant according to the invention additionally is prevented from a rotation about the optical axis of the eye.

If the surface structure is toothed and/or undulating, the implant according to the invention is provided with a surface structure of the support element, which is particularly advantageous for the form-fit of the support element with the ciliaris sulcus.

In a preferred embodiment of the implant according to the invention, the implant has holding means, wherein the holding means are designed for holding at least one sensor and/or a telemetry unit in the holding plane and are connected to the deflecting element. The holding means may be, for example, a perforated plate, to which, for example, a pressure sensor for measuring the intraocular pressure and/or a sensor for measuring the glucose level and/or a temperature sensor for temperature measurements can be fixed by means of, for example, an adhesive connection. The transmission of the data measured by the sensor can take place via an induction coil of a telemetry unit to an external receiver, which is located outside the eye. Advantageously, the implant according to the invention can be used with the aid of the holding means for monitoring clinical parameters of the eye such as, for example, intraocular pressure.

Preferably, in the implant according to the invention the holding means and the support element may be arranged spaced from each other with respect to the normal of the deflection plane. Advantageously, for a trauma-free implanting, the holding means, such as a plate provided for holding a pressure sensor and an induction coil, may be positioned with a rail frame, as far away as possible from the preferred site of the implant, usually near the iris, with respect to the optical axis of the eye. The risk that, for example, the pressure sensor of the holding means collides with the iris, may thus be reduced with the aid of the implant according to the invention. This is due to the fact that the fixing site, for example, the ciliaris sulcus, in the implant according to the invention, is spatially separated from the holding means which in the implant according to the invention lie in a holding plane, which is arranged with respect to the optical axis of the eye further away from the iris than the support element, along the optical axis of the eye in the implanted state. Thus, the implant according to the invention can advantageously be fixed both in the posterior chamber of the eye, namely in the ciliaris sulcus, as well as in the anterior chamber of the eye, namely in the iridocorneal chamber angle, in order to prevent injury to the iris during implant.

In a development of the invention, the deflecting element lies in at least one portion in an inclination plane, the inclination plane and the deflection plane being angled relative to each other. The portion of the deflecting element, such as the central portion of a wire-shaped compression spring, thus protrudes out of the deflection plane in the implant according to the invention due to the inclination plane and deflection plane forming an angle with each other. In this way, in the implant according to the invention, a particularly advantageous spatial separation is implemented in a simple manner between the holding means, which interact with the deflecting element, such as a compression spring, for example a plate glued to one end of a compression spring, and the deflection plane, such as the plane lying in the furrow bottom of the ciliaris sulcus, so that the risk of collision between a sensory unit fastened to the holding means of the implant according to the invention and the iris may be minimised.

Preferably, in the implant according to the invention, in the implanted state, the holding means may be positioned at a distance from the optical axis of the eye in a direction transverse to the optical axis of the eye. Advantageously, in the implant according to the invention, the holding means provided with a plate are positioned outside the optical axis of the eye, for example, so that a shadowing of the optical axis of the eye, which may impair the vision of the patient, may be avoided.

In a preferred embodiment of the implant according to the invention, the fixing means may be fastened by means of releasable fastening means. ‘Releasable fastening means’ in the sense of the invention are for example locking elements, so that the fixing means, which are formed, for example, by a flexible wire, lock at one end of the wire into the holding means, which are formed by a frame element, for example. The implant according to the invention may thus be modularly composed of fixing means, such as a flexible wire, and the holding means, such as a frame. Advantageously, with respect to a minimally invasive implant, the modules, namely the fixing means and the holding means, may be implanted independently of one another, in order to connect them intraocularly by means of the locking elements, thus allowing, for example, for smaller surgical cuts for accessing the anterior chamber of the eye, with respect to known implants.

Finally, in a further advantageous embodiment of the implant according to the invention, the holding means are formed integrally with the sensor and/or the telemetry unit, in the form of a polymeric casting. Thus, the implant according to the invention may be advantageously embedded, in a minimally invasive implantation, into a silicone rubber matrix, for example.

The invention is described by way of example in the following in a preferred embodiment with reference to the drawings, wherein further advantageous details may be obtained from the drawings.

Functionally identical parts are provided with the same reference numerals.

In detail, in the drawings:

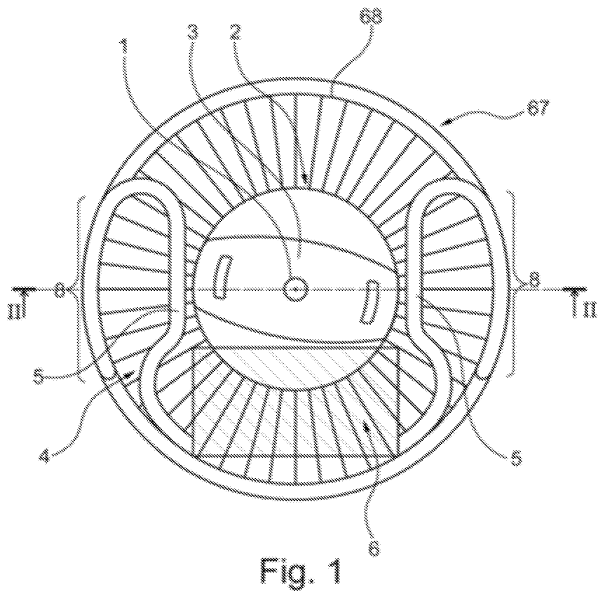

FIG. 1 is a plan view in the direction along the optical axis of an eye on an implant implanted in an eye implant according to a preferred embodiment of the invention;

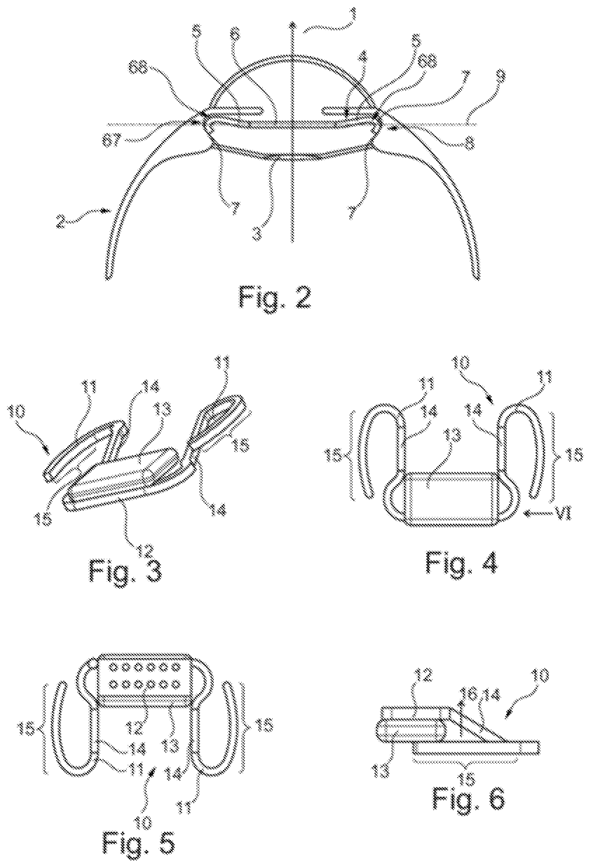

FIG. 2 is a section of the implant according to FIG. 1 along the line II of FIG. 1;

FIG. 3 sis a perspective view of an implant according to the invention in a further preferred embodiment;

FIG. 4 is a plan view from above of the implant of FIG. 3;

FIG. 5 is a plan view from below of the implant of FIGS. 3 and 4;

FIG. 6 is a lateral view of the implant of FIGS. 3 to 5 in the direction of arrow VI of FIG. 4;

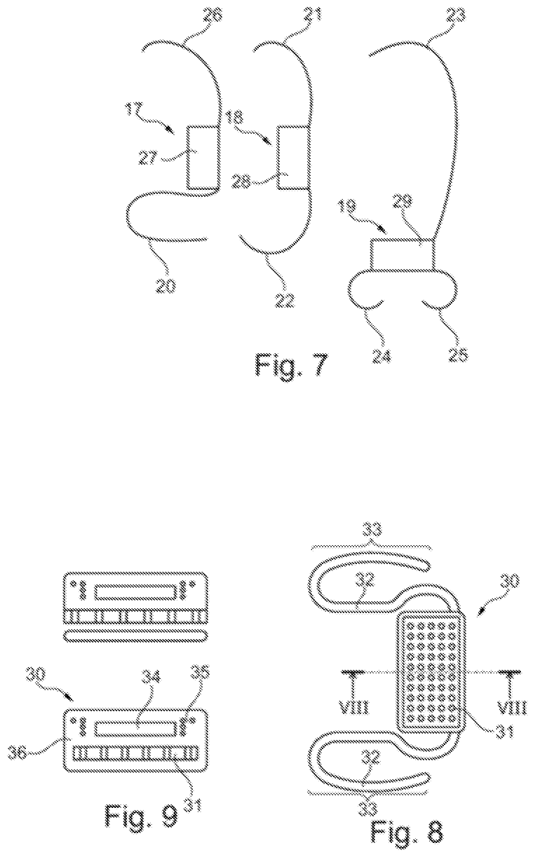

FIG. 7 is a schematic representation of three implants according to the invention in a further preferred embodiment;

FIG. 8 is a schematic plan view of an implant according to the invention in a further preferred embodiment;

FIG. 9 is a sectional view of the implant according to FIG. 8 along the line IX according to FIG. 8;

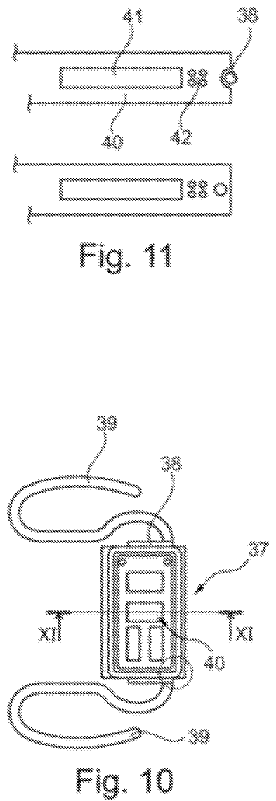

FIG. 10 is a schematic plan view of an implant of the invention in a further preferred embodiment;

FIG. 11 is a sectional view of the implant of FIG. 10 along line XI of FIG. 10;

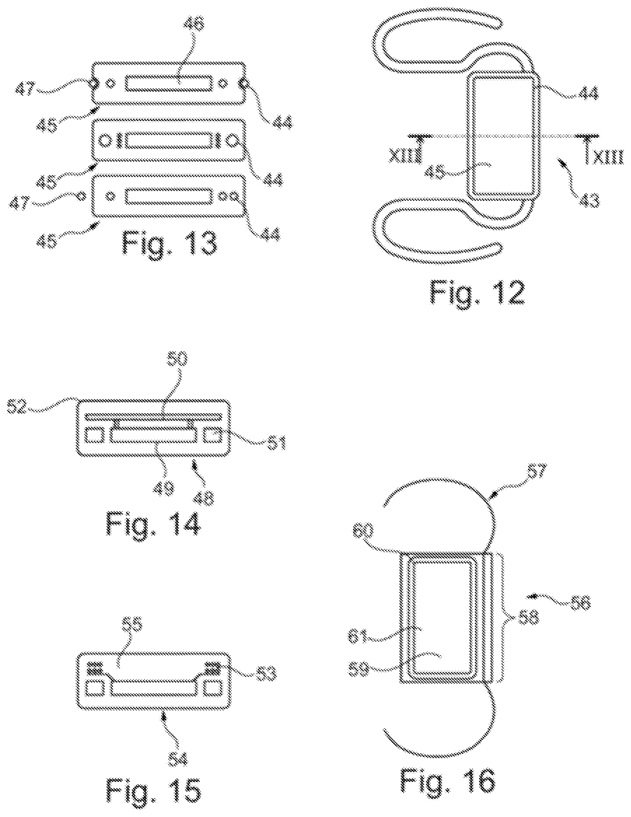

FIG. 12 is a schematic view from above of an implant of the invention in a further preferred embodiment;

FIG. 13 is a sectional view of implant of FIG. 12 along line XIII in FIG. 12;

FIG. 14 is a sectional view of an implant of the invention in a further preferred embodiment;

FIG. 15 is a sectional view of an implant of the invention in a further preferred embodiment;

FIG. 16 is a schematic plan view of an implant of the invention in a further preferred embodiment; and

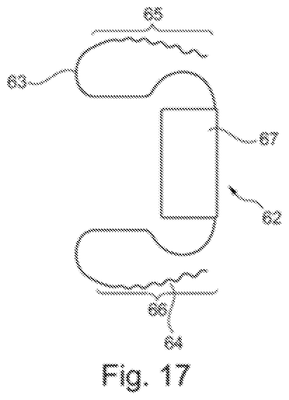

FIG. 17 is a schematic plan view of an implant according to the invention in a further preferred embodiment.

FIG. 1 is, in the viewing direction along the optical axis 1 of an eye 2, a plan view of the eye 2 with an artificial lens 3 and an implanted implant 4 according to a preferred embodiment of the invention. The implant 4 consists of two bent compression springs 5 and a plate 6 which is integrally formed with the compression springs 5, which plate is connected via an adhesive connection with a sensor telemetry module (both not shown) for measuring the intraocular pressure and for transmitting data regarding the intraocular pressure to an external receiver.

It can be seen from FIG. 1 that the compression springs 5 press the implant 4 against a furrow bottom 7 of the ciliaris sulcus 68, so that the implant 4 is fixed transversely to the direction of the optical axis 1 via a bow portion 8 to the furrow bottom 7 via a biasing force of the compression springs 5. The plate 6 is arranged in the direction transverse to the optical axis 1 outside of the artificial lens 3 in order to avoid shading of the artificial lens 3 negatively influencing the eyesight. FIG. 2 is a sectional view of the implant 4 from FIG. 1. With reference to FIG. 2, it is clear that the compression springs 5, in case of an elastic deformation in a plane 9, which is perpendicular to the optical axis 1, recover their shape such that the bow portion 8 of the compression springs 5 is pressed against the furrow bottom 7 of the ciliaris sulcus 68, whereby the implant 4 is fixed transversely to the direction of the optical axis 1.

FIGS. 3 to 6 are different representations of a further implant 10 according to the invention. Similarly to the implant 4 of FIGS. 1 and 2, the implant 10 according to FIGS. 3 to 6 is composed of two compression springs 11 and a plate 12 which is integrally formed with the compression springs 11. The plate 12 is connected via an adhesive connection (not shown) to a sensor telemetry module 13. Liquid adhesive has flowed into the holes of the plate 13 shown in FIG. 5, so that the sensor telemetry module 13 is additionally anchored to the plate 13 via the adhesive which subsequently hardens within the holes.

It is clear from the illustration of the implant 10 in FIGS. 3 and 6 that the compression springs 11 have an angled middle portion 14. As a result, bow portions 15 of the compression springs 11 resting on the furrow bottom of the ciliaris sulcus, not shown in FIGS. 3 to 6, are spatially separated from the plate 12 and thus from the sensor telemetry modules 13 along a direction 16 shown in FIG. 6. As a result of this spatial separation, due to the angled portion 14 of the compression springs 11 in the implanted state of the implant 10, it is ensured that the risk of a collision of the plate 12 and/or the sensor telemetry module 13 with the iris of an eye is minimised.

This is due to the fact that the implant 10 can be fixed, via the bow portions 15, due to the elastic shape recoverability of the compression springs 11, in such a way that the bow portions 15 are arranged nearer to the iris along the optical axis of the eye (cf. FIG. 2), than the plate 12 or the sensor telemetry module 13 of implant 10. Similarly, the implant 10 may also be fixed in the anterior chamber of the eye, due to the angled portion 14 in the iridocorneal chamber angle, so that also in this implantation, the risk of collision with the iris is minimised due to the fact that the plate 12 or the sensor telemetry module 13 is spaced further apart from the iris than the bow portions 15.

FIG. 7 is a schematic representation of three implants 17, 18 and 19 according to the invention, each in a further preferred embodiment. The implant 17 has, in addition to a plate 27, compression springs 20 and 26, the elastic shape recovery of which, according to the implant of FIGS. 1 and 2 and FIG. 3-6 causes a fixing of the implant 17 in the furrow bottom of the ciliaris sulcus. The mean radius of curvature of the compression spring 26 is greater than the mean radius of curvature of the compression spring 20; it is thereby possible that the plate 27 provided for a sensor telemetry module can be positioned as far as possible outside the optical axis in the implanted state. In an analogous manner, the implant 18 is composed, in addition to a plate 28, of a compression spring 21 and a compression spring 22, wherein the mean radius of curvature of the compression spring 21 is greater than the mean radius of curvature of the compression spring 22. Compared to the compression springs 20 and 26 of the implant 17, however, the difference between the radii of curvature of the compression springs 21 and 22 is lower. Thus, also in the implant 18 it is ensured that the plate 28 provided for a sensor telemetry module can be positioned as far as possible outside the optical axis in the implanted state. The implant 19, however, is composed, in addition to a plate 29, of three compression springs 23, 24 and 25. The compression springs 24 and 25 are arranged opposite to the compression spring 23. The radius of curvature of the compression spring 23 is greater than the radius of curvature of the compression springs 24 and 25. Thus, also in the implant 19 it is ensured that the plate 28 provided for a sensor telemetry module can be positioned as far as possible outside the optical axis in the implanted state.

FIG. 8 is a representation of an implant 30 in a further preferred embodiment according to the invention. The implant 30 consists of a perforated plate 31 and compression springs 32 having bow portions 33 provided for resting on the furrow bottom of the ciliaris sulcus and which may be deflected by an elastic shape recovery of the compression springs 32 in the direction of the ciliaris sulcus.

FIG. 9 is a sectional view of the implant 30 from FIG. 8. The sectional view of FIG. 9 shows that, in the case of the implant 30, a pressure sensor 34, together with an induction coil 35 which telemetrically transmits measurement data acquired by the pressure sensor to a receiver, is surrounded by a polymer matrix 36, so that the pressure sensor 34, the induction coil 35 and the plate 31 are provided as a one-piece component in the implant 30. The polymer matrix 36 could flow in the liquid state into the holes in the plate 31, whereby the pressure sensor 34 and the induction coil 35 are anchored to the plate 31 via the polymer matrix 36 in the holes in the plate 31. In this case, the polymer matrix 36 consists of silicone rubber.

FIG. 10 is a schematic plan view of a further implant 37 according to the invention. According to FIG. 10, the implant 37 consists of compression springs 39 shaped analogously to the implants from FIGS. 1 to 9 and a frame 38 which is open on one side and in which, according to the sectional view of FIG. 11, a pressure sensor 41 embedded in a polymer matrix 40 made of silicon rubber and including an induction coil 42 is supported. The polymer matrix 40 has therefore been introduced into the frame 38 on the open side of the frame 38. The implant 37 is thus modular, namely a module consisting of the compression springs 39 and the frame 38 and a module removable from the frame 38 consisting of the polymer matrix 40 made of silicone rubber comprising the pressure sensor 41 and the induction coil.

FIG. 12 is a schematic plan view of a further implant 43 according to the invention having a frame 44 which, in contrast to the frame 38 of the implant 37 according to FIGS. 10 and 11, is circumferentially closed. In the frame 44 of the implant 43, similar to the implant 37 from FIGS. 10 and 11, a polymer matrix 45 is mounted in which a pressure sensor 46 and an induction coil 47 are embedded. The sectional representation of the implant 43 from FIG. 13 shows that the polymer matrix 45 is mounted in a form-fitting manner in the frame 44 of the implant 43 by means of a groove (FIG. 13, above), or the frame 44 is completely embedded in the polymer matrix 42 (FIG. 13, centre), or the frame 44 is embedded in part in the polymer matrix 45 (FIG. 13, below) by placing a rail 47 of the frame outside the polymer matrix (FIG. 13, below).

FIG. 14 is a sectional view of an implant 48, in which a pressure sensor 49 and a planar coil 50 serving as a telemetry unit are embedded together with a holding frame 51 in a polymer matrix 52.

FIG. 15 is a sectional view of an implant 54 designed analogously to the implant 48 from FIG. 14, but in which a coil 53 is embedded in a polymer matrix 55.

FIG. 16 shows in plan view a schematic representation of an implant 56 according to the invention, in which a one-piece compression spring 57, which consists of a thread, is embedded on a portion 58 into a polymer matrix 59 together with a coil 60 and a pressure sensor 61. The implant 56 therefore has a particularly compact construction, which is thus advantageous for implanting.

The plan view in FIG. 17 of a schematically illustrated implant 62 shows that the implant 62 consists of plate 67 and two springs 63 and 64. The spring 63 has, in a bow portion 65, an undulating structure which as a result is suitable for resting on the ciliaris sulcus having membranous segmentations. By contrast, the spring 64 of the implant 62 has a tooth-shaped structure in a bow portion 66, which structure is particularly well suited for fixing the implant 62 for preventing it from rotating about an optical axis (not shown) of the eye.

### LIST OF REFERENCE NUMERALS

1 optical axis
2 eye
3 lens
4 implant
5 compression spring
6 plate
7 furrows bottom
8 bow section
9 plane
10 implant
11 compression spring
12 plate
13 sensor telemetry module
14 middle portion
15 bow portion
16 direction
17-19 implant
20-26 compression spring
27-29 plate
30 implant
31 plate
32 compression springs
33 bow portion
34 pressure sensor
35 induction coil
36 polymer matrix
37 implant
38 frame
39 compression spring
40 polymer matrix
41 pressure sensor
42 induction coil
43 implant
44 frame
45 polymer matrix
46 pressure sensor
47 rail
48 implant
49 pressure sensor
50 planar coil
51 holding frame
52 polymer matrix
53 coil
54 implant
55 polymer matrix
56 implant
57 compression spring
58 portion
59 polymer matrix
60 coil
61 pressure sensor
62 implant
63-64 spring
65-66 bow portion
67 plate
68 ciliaris sulcus

1. A method comprising:
implanting in and/or on an eye an implant with a fixing apparatus, characterised in that the fixing apparatus comprises at least one support element which is mounted on a tissue structure in a direction transverse to an optical axis of the eye.

2. The method of claim 1, characterised in that the fixing apparatus has at least one deflecting element for deflecting the support element in a deflection plane, wherein the deflection plane is positioned in an implanted state perpendicular to the optical axis of the eye.

3. The method of claim 2, characterised in that the at least one deflecting element is able to recover its shape after deformation of the deflecting element, wherein the deflection is based on the shape recoverability.

4. The method of claim 3, characterised in that the shape recoverability is elastic.

5. The method of claim 3, characterised in that the shape recoverability is stimulated via a stimulus.

6. The method of claim 5, characterised in that the stimulus is a variation of temperature.

7. The method of claim 2, characterised in that the at least one deflecting element and/or the support element comprises a polymer.

8. The method of claim 2, characterised in that the at least one deflecting element and/or the support element comprises, at least in portions, of a material having shape memory properties.

9. The method of claim 2, characterised in that the at least one deflecting element in a direction perpendicular to the deflection plane has a shape which is rigid with respect to dents

10. The method of claim 2, characterised in that the at least one deflecting element and/or the support element are configured arcuate at least in portions.

11. The method of claim 2, characterised in that the deflecting element lies in at least one portion in an inclination plane, the inclination plane and the deflection plane being angled relative to one another.

12. The method of claim 2, characterised in that that the implant comprises a holding apparatus, wherein the holding apparatus is configured for holding at least one sensor and/or a telemetry unit in a mounting plane and are in operative connection with the at least one deflecting element.

13. The method of claim 12, characterised in that the holding apparatus and the support element is arranged spaced from each other with respect to the normal of the deflection plane.

14. The method of claim 12, characterised in that, in the implanted state, the holding apparatus is arranged in the direction transverse to the optical axis of the eye at a distance from the optical axis of the eye.

15. The method of claim 12, characterised in that the fixing apparatus is fastened to the holding apparatus by a releasable fastening.

16. The method of claim 12, characterised in that the holding apparatus is integrally formed with the sensor and/or the telemetry unit as a polymer casting.

17. The method of claim 1, characterised in that the support element has a segmented surface structure on a surface provided for contacting with the tissue structure.

18. The method of claim 17, characterised in that the segmented surface structure is tooth-shaped and/or undulating.

19. An implant comprising:
a sensor for measuring intraocular pressure;
a telemetry unit for transmitting the measured signal detected by the sensor;
at least one support element, which is mounted on a tissue structure in a direction transverse to an optical axis of an eye; and
at least one deflecting element for deflecting the support element in a deflection plane, the deflection plane being positioned in an implanted state perpendicular to the optical axis of the eye.

20. A method comprising:
implanting in and/or on an eye an implant with a fixing apparatus, characterised in that the fixing apparatus comprises at least one support element which is mounted on a tissue structure in a direction transverse to an optical axis of the eye;
deflecting the support element in a deflection plane with at least one deflecting element, wherein the deflection plane is positioned in an implanted state perpendicular to the optical axis of the eye; and
holding at least one sensor and/or a telemetry unit in a mounting plane with a holding apparatus, wherein the at least one sensor and/or the telemetry unit are in operative connection with the deflecting element.
